(19) United States
(12) Patent Application Publication
(10) Pub. No.: US 2010/0249593 A1
(43) Pub. Date: Sep. 30, 2010

(54) ULTRASOUND DIAGNOSTIC APPARATUS AND METHOD, AND COMPUTER PROGRAM PRODUCT

(75) Inventors: Tomoyuki Takeguchi, Kanagawa (JP); Masahide Nishiura, Tokyo (JP); Yukinobu Sakata, Tokyo (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: KABUSHIKI KAISHA TOSHIBA, Tokyo (JP)

(21) Appl. No.: 12/561,786

(22) Filed: Sep. 17, 2009

(30) Foreign Application Priority Data
Mar. 26, 2009 (JP) 2009-077306

(51) Int. Cl. A61B 8/14; G06F 3/048

(52) U.S. Cl. 600/443; 715/771

(57) ABSTRACT

An ultrasound diagnostic apparatus includes a first storage unit configured to store three-dimensional image data acquired by measuring the organ; a second storage unit configured to store a region name in association with a display method, the region name being the name of a region of the organ and the display method being for displaying the region of the organ; a region retrieval unit configured to retrieve, based on the region name, the region from the three-dimensional image data; and a display unit configured to display a two-dimensional image data; and a display control unit configured to transform, based on the region retrieved by the region unit, the three-dimensional image data of the region into the two-dimensional image for the region and configured to display the two-dimensional image on the display unit using the display method.

### CROSS-REFERENCE TO RELATED APPLICATIONS

This application is based upon and claims the benefit of priority from the prior Japanese Patent Application No. 2009-077306, filed on Mar. 26, 2009; the entire contents of which are incorporated herein by reference.

### BACKGROUND OF THE INVENTION

1. Field of the Invention

The present invention relates to an ultrasound diagnostic apparatus and method, and a computer program product.

2. Description of the Related Art

In a medical examination of cardiac function by use of a two-dimensional ultrasound diagnostic apparatus, representative sectional images such as apical 4 chamber view or apical 2 chamber view are required. In order to acquire such representative sectional images, a user adjusts the position or angle of a probe (or a transducer) so as to obtain a required section, while viewing the picture of a sectional image just taken.

On the other hand, to measure a heart by use of a three-dimensional ultrasound diagnostic apparatus, volume data, which is three-dimensional image data including the whole heart, is photographed. Then, in order to display a representative image among a number of sectional images defined within the same volume data, a user adjusts a sectional image position so that a requested sectional image is displayed. Such an adjustment is made using, for example, a position change knob for moving the sectional position of volume data in a parallel direction or an angle adjustment knob for rotating the section three-dimensionally.

The foregoing method requires a user to perform a tangled operation to determine a section of three-dimensional volume data in order to acquire a required sectional image suitable for the user to observe a target region. Where more than one section is necessary, further operations are required. To overcome the foregoing problem, the following technical document discloses a proposal to acquire a representative sectional image automatically: “AUTOMPR: Automatic detection of standard planes in 3D echocardiography” by Lu x, et. al, 5^{th} IEEE Intl. Symposium on Biomedical Imaging, 2008.

However, in the method descried in this technical document, a user has to translate a target observation region into a representative sectional image based on his or her knowledge of how to determine a representative image among a number of sectional images that include the target observation region. This method accordingly does not allow for intuitive operation. In addition, there are limits to representative sectional images; therefore, depending on the target observation region, it may be difficult to acquire an appropriate image for observing or examining this region.

### SUMMARY OF THE INVENTION

According to one aspect of the present invention, an ultrasound diagnostic apparatus includes a first storage unit configured to store three-dimensional image data acquired by measuring the organ; a second storage unit configured to store a region name in association with a display method, the region name being the name of a region of the organ and the display method being for displaying the region of the organ; a region retrieval unit configured to retrieve, based on the region name, the region from the three-dimensional image data; and a display unit configured to display a two-dimensional image data; and a display control unit configured to transform, based on the region retrieved by the region unit, the three-dimensional image data of the region into the two-dimensional image for the region and configured to display the two-dimensional image on the display unit using the display method.

According to another aspect of the present invention, an ultrasound diagnostic method, includes retrieving, based on a region name which is the name of a region of an organ, the region from three-dimensional image data about the organ, the three-dimensional image data being acquired by measuring the organ; and transforming, based on the retrieved region, the three-dimensional image data of the region into a two-dimensional image for the region; and displaying the two-dimensional image on a display unit using a display method corresponding to the region.

According to still another aspect of the present invention, a computer readable product having a computer readable medium including programmed instructions. The instructions, when executed by a computer, cause the computer to perform retrieving, based on a region name which is the name of a region of an organ, the region from three-dimensional image data about the organ, the three-dimensional image data being acquired by measuring the organ; and transforming, based on the retrieved region, the three-dimensional image data of the region into a two-dimensional image for the region; and displaying the two-dimensional image on a display unit using a display method corresponding to the region.

### BRIEF DESCRIPTION OF THE DRAWINGS

FIG. 1 is a block diagram of an ultrasound diagnostic apparatus according to a first embodiment;

FIG. 2 is a memory structure for a display storage area;

FIG. 3 shows the relation between volume data and sectional images;

FIG. 4 is a flowchart for the ultrasound diagnostic process according to the first embodiment;

FIG. 5 is a block diagram of an ultrasound diagnostic apparatus according to a second embodiment; and

FIG. 6 is a flowchart for the ultrasound diagnostic process according to the second embodiment.

### DETAILED DESCRIPTION OF THE INVENTION

Referring to the accompanying drawings, hereinafter will be described in detail an ultrasound diagnostic apparatus and method, and a computer program product according to exemplary embodiments of the present invention.

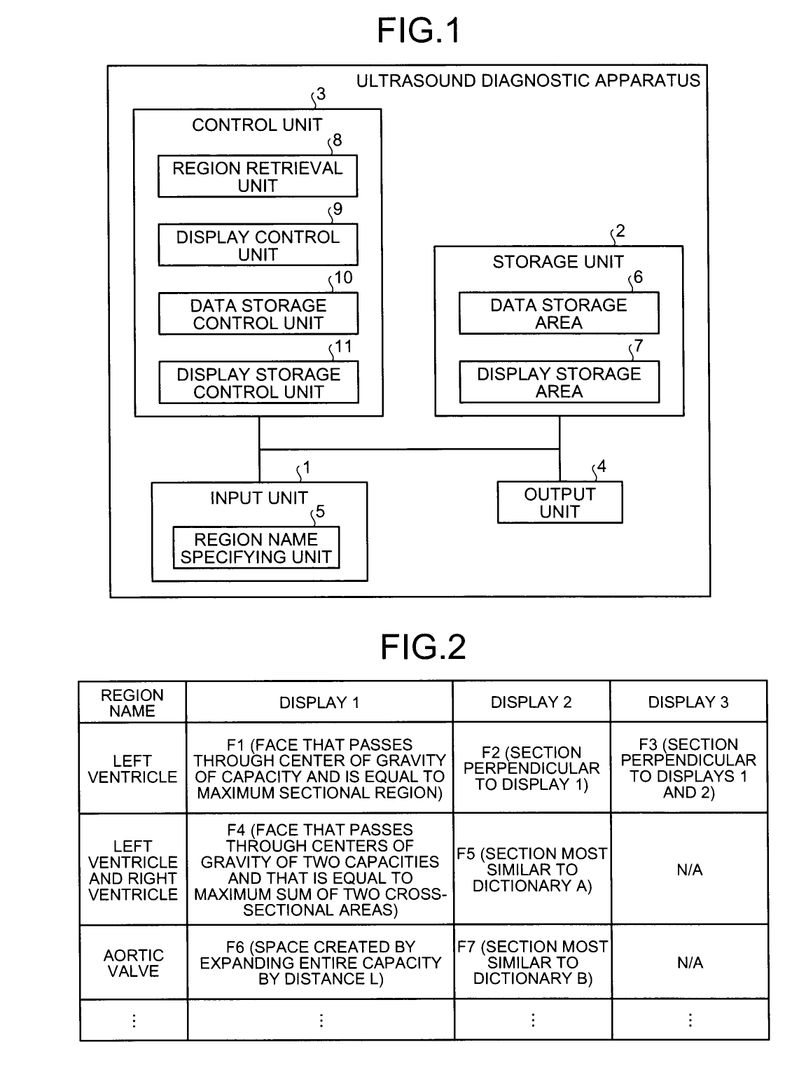

As shown in FIG. 1, an ultrasound diagnostic apparatus according to a first embodiment includes an input unit 1, a storage unit 2, a control unit 3, and an output unit 4. These units are coupled so as to communicate with one another, and are connected by circuits that mutually exchange control signals, information, etc.

The input unit 1 receives information input from an operator that operates the ultrasound diagnostic apparatus. The input unit 1 includes a region name specifying unit 5. The region name specifying unit 5 receives a target region name specified by the operator. The region name specifying unit 5 may be composed of, for example, a push button disposed on the diagnostic apparatus, or may be a virtual button shown on a touch panel or display. Alternatively, the input unit 1 may incorporate a microphone and use voice recognition technology, thereby selecting the region name specified by the voice of an operator.

The storage unit 2 stores therein various data. The storage unit 2 may be a general medium, such as a semiconductor memory or a magnetic disk, which is able to store information. The storage unit 2 includes a data storage area 6 and a display storage area 7. The data storage area 6 stores volume data, i.e., three dimensional image data of a photographed organ. For example, where a heart is measured using an ultrasound diagnostic apparatus, the data storage area 6 stores the volume data including the photographed entire heart.

The display storage area 7 stores the region name in association with a display method specified during the selection of the region for volume data on the output unit 4. The term “display method” refers to the method for forming an image of the region from the corresponding volume data. In an example of a storage structure for the display storage area 7 as shown in FIG. 2, a table structure has stored therein each region name in association with a plurality of display methods.

For example, where the region name “left ventricle” is entered as an index key, a display control unit 9 in the control unit 3 described below may acquire F1 (a face that passes through the center of gravity of the capacity and is equal to the maximum sectional region) as the first display method (display 1). For the region name “left ventricle,” the following display methods region are additionally stored: F2 (a section perpendicular to the display 1 “F1”); and F3 (a section perpendicular to the display 1 “F1” and the display 2 “F2”). Accordingly, the display control unit 9 can acquire a plurality of display methods including the F2 (display 2) and F3 (display 3) as the second and third display methods.

As described above, each display method obtained in the display storage area 7 is a rule describing how to form an image to be displayed on the output unit 4. It is preferable to set in advance a rule described in the table. However, an empty table may be provided for storing a rule when it is to be determined by an operator. Alternatively, the table may be designed such that an operator may change, add, or delete rules stored in the display storage area 7.

If “the left ventricle” and “the right ventricle” are specified by the region name specifying unit 5, the display control unit 9 can acquire F4 (a face that passes through the centers of gravity of the two capacities and that is equal to the maximum sum of the two cross-sectional areas) as the first display method. For the region names, “the left ventricle” and “the right ventricle,” the display control unit 9 additionally stores F5 (a section most similar to a dictionary A) as the second display method. The dictionary A refers to a recognition dictionary for use in the determination of a section that is formed from, for example, an image for learning, and that is optimal for diagnosis. This dictionary A is stored in the display storage area 7.

The images of regions formed by these display methods are not limited to sectional images. For example, if an aortic valve is specified by the region name specifying unit 5, the display control unit 9 can acquire F6 (a space created by expanding the entire capacity by distance L) as the first display method. In view of the degree of ease of medical examination, the direction in which a valve is observed may be limited in some degrees. Therefore, a rule for determining viewing direction may be described together with the rule for determining a space. For instance, the position of a detected aorta is planar-approximated and the direction perpendicular to the plane as viewed from the left ventricle side may be referred to as the viewing direction.

Next will be described a case where the display control unit 9 acquires F7 (the section most similar to a dictionary B) as the second display method. The dictionary B refers to a recognition dictionary for use in the determination of a section that is formed from, for example, an image for learning, and that is optimal for diagnosis. This dictionary B is stored in the display storage area 7. In F1 and the others, a cut face is defined by the capacity or shape of a specified region. However, a region such as a valve ring in the aorta is a thin film and it is difficult to determine a single sectional image from its capacity or shape. For this reason, a rule for determining a cut face that displays a region is to employ a sectional image most similar to the dictionary B among sections that include a specified region.

A control unit 3 controls the overall ultrasound diagnostic apparatus and performs various data processing, operation, or calculation. The control unit 3 includes, in addition to a region retrieval unit 8, the display control unit 9, a data storage control unit 10, and a display storage control unit 11. The control unit 3 is, for example, a processor.

The region retrieval unit 8 performs a calculation such that a region corresponding to the region name received by the region name specifying unit 5 is located in volume data stored in the data storage area 6. A method for retrieving an region name in the volume data by means of a calculation may use, for example, the technology described in “Real-Time Tracking of the Left Ventricle in 3D Echocardiography Using a State Estimation Approach,” by Fredric Orderud, Joger Hansgard, and Stein I. Rabben, 10^{th} International Conference on Medical Image Computing and Computer Assisted Intervention (MICCAI 2007).

The display control unit 9 exerts control as follows: the display control unit 9 captures volume data stored in the data storage area 6, a display method for an region stored in the display storage area 7, and information about the region retrieved by the region retrieval unit 8; following the display method for the region, the display control unit 9 then forms a two-dimensional display image based on both the volume data and the information about the region, and outputs this image to the output unit 4.

The data storage control unit 10 exerts control so as to store three-dimensional volume data into the data storage area 6 of the storage unit 2. The display storage control unit 11 exerts control so as to store region name into the display storage area 7 of the storage unit 2 while associating the same with a display method (specified during the selection of the region name) for the volume data on the output unit 4.

The output unit 4 provides an operator with the output (i.e., display) of various information, such as two-dimensional display images. The output unit 4 may be, for example, a display apparatus.

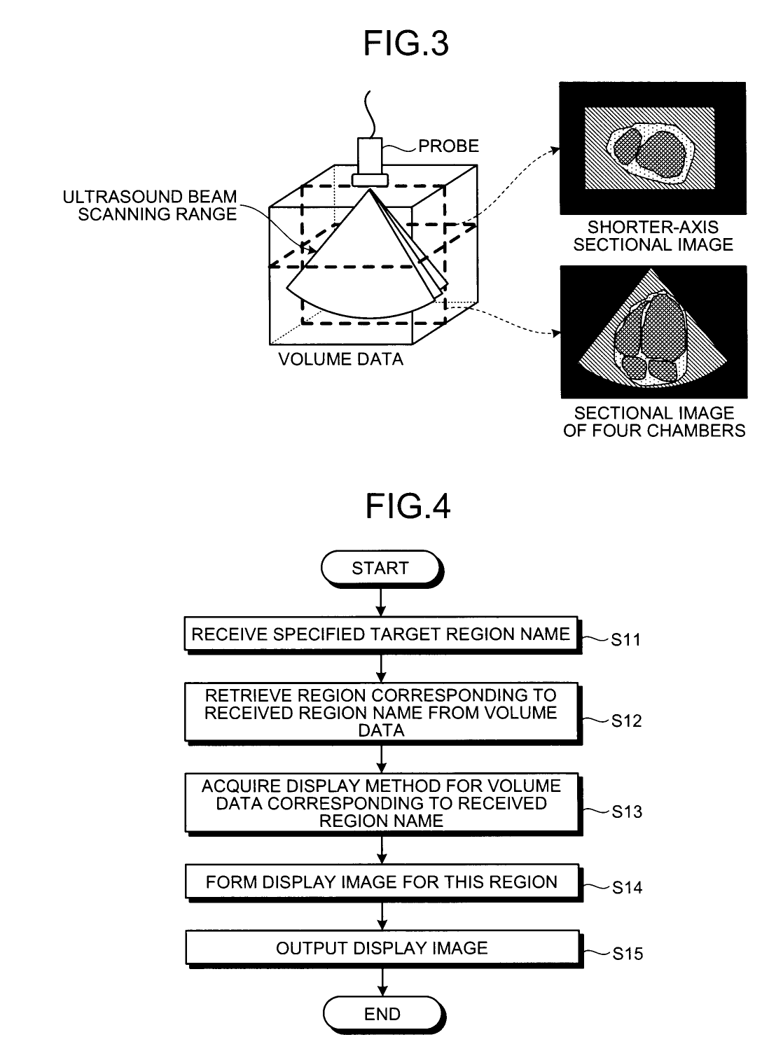

FIG. 3 shows the relation between the volume data and the sectional images. As shown in FIG. 3, three-dimensional volume data is captured within the scanning range of an ultrasound beam generated by a probe. The three-dimensional volume data shows sectional images that are quite different depending on a cut face or a displayed space. FIG. 3 shows an example where a shorter-axis sectional image and a sectional image of the four chambers region are acquired. Accordingly, to manually retrieve from volume data an image of the target region of an organ being examined requires a vexing and complex operation. In view of such problems, the ultrasound diagnostic apparatus according to the present embodiment of the present invention makes it possible to observe, simply by an operator's specifying the name of an region, an optimal image that includes this region.

Referring to FIG. 4, next will be described the operation of an ultrasound diagnostic apparatus according to the first embodiment. First, the region name specifying unit 5 receives a specified target region name from an operator (step S11). For example, where the region name specifying unit 5 includes a push button, the region name is specified by the operator's depressing the push button. In this case, in order to improve the user interface, the region name specifying unit 5 may be designed such that if an operator selects a certain region name that has been specified, this selection may be canceled.

Alternatively, the region name specifying unit 5 may be designed such that among presented region names, a plurality of region names that can be specified simultaneously may be limited to one or to two or more. In a region name specifying unit 5 designed to enable simultaneous specification of two or more region names, a push button, command, or other instruction may be provided to allow cancellation of all specified region names.

The region names that can be specified vary from organ to organ. The present embodiment has been described using as an example the case where the organ to be examined is the heart. In this case, region names that may be specified are, for example, the left ventricle, right ventricle, left atrium, right atrium, aorta, tricuspid valve, pulmonary valve, mitral valve, aortic valve, interventricular septum, interatrial septum, etc. The region to be examined is closely related to a specific organ; generally the region name to be specified differs according to the organ to be examined. The region name specifying unit 5 may, therefore, include an organ specifying section that specifies the organ to be examined. Where an organ specifying section is included, region names displayed by the region name specifying unit 5 may be changed automatically according to the organ specified.

Subsequently, the region retrieval unit 8 performs a calculation, thereby retrieving from the volume data stored in the data storage area 6 a region name specified by the region name specifying unit 5 (step S12). In the present embodiment, a description is given using as an example a case where the region name specified by the region name specifying unit 5 is the left ventricle. It is, therefore, possible to find where information about a part or space corresponding to the left ventricle specified by the region name specifying unit 5 is located in the volume data.

Subsequently, the display control unit 9 acquires a display method for the volume data corresponding to the region name received by the region name specifying unit 5 (step S13). In this embodiment, the display control unit 9 selects a display method for the volume data corresponding to the left ventricle.

Next, the display control unit 9 acquires volume data stored in the data storage area 6, the display method stored in the display storage area 7, and information about the region retrieved by the region retrieval unit 8, and forms a display image for this region according to the display method (step S14).

In this embodiment, where the left ventricle is specified, the rule F1 is assigned to the first display method. The display control unit 9, therefore, forms a display image for the cut face of the left ventricle the area of which is the largest among planes (retrieved by the region retrieval unit 8) that include the center of gravity of the left ventricle. A method for determining the largest cut face may include forming sectional images obtained by a number of cut faces each of which includes the center of gravity of the left ventricle, and then selecting the face that has the largest area among the sectional images of the left ventricle. After the cut face is determined, the direction of the X-axis of the coordinate system for the volume data may be used as the direction of the X-axis for the image for display. Furthermore, based on the rules F2 and F3 assigned to the second and third display methods respectively, the display control unit 9 forms the corresponding display images.

Lastly, the display control unit 9 outputs (i.e., displays) the display images thus formed onto the output unit (step S15). Where a plurality of display methods are stored in the display storage area 7, a plurality of images are formed for display by the display control unit 9. In this case, the display control unit 9 may cause the output unit 4 to display all these images simultaneously. Alternatively, the display control unit 9 may cause the output unit 4 to select and display one of the images thus formed following an instruction given by a display switching button provided for the input unit 1. In the present embodiment, three rules, i.e., F1, F2, and F3, are assigned to the display method for the left ventricle, and each of them is displayed on the output unit 4 as requested.

Thus, when an operator specifies a target region name for observation, the optimal image including this region is shown on the output unit 4.

As described above, the ultrasound diagnostic apparatus according to the first embodiment functions even when a plurality of specifiable region names are selected simultaneously. A description is given of the case where, for example, the region name specifying unit 5 selects both the left and right ventricles. The display storage area 7 stores region names and display methods (selected along with the region names) for volume data on the output unit 4 so that connections are made between them. Specifically, as shown in FIG. 2, the display storage area 7 stores, as the region names, three names of the region names “left ventricle,” “left ventricle and right ventricle,” and “right ventricle (not shown).” In this case, the region name retrieve unit 8 retrieves pieces of information about the region of the left and right ventricles from the volume data in the data storage area 6. Then, following display methods for “the left ventricle and right ventricle,” the display control unit 9 forms display images.

Therefore, the number of the selections of region names made by the region name specifying unit 5 equals the number of combinations of specifiable region names. However, a combination of region names difficult to observe simultaneously within a single section does not have to be included. In order to provide a more desirable interface, the region name specifying unit 5 may be designed to inhibit a user's selection of any combination of region names that cannot be provided with display methods in the display storage area 7. For example, if the display storage area 7 does not contain a rule corresponding to the simultaneous selection of the right ventricle and aorta, the selection of the aorta is nullified after selection of the right ventricle.

Where a plurality of region names are simultaneously selected, display images may be formed following all the rules stored in the display storage area 7. Specifically, if the left and right ventricles are selected by the region name specifying unit 5, the display control unit 9 may form display images following the respective rules of the three display methods corresponding to the region names “left ventricle,” “right ventricle,” and “left and right ventricles.”

The ultrasound diagnostic apparatus according to the first embodiment functions even if the storage unit 2 does not have the display storage area 7. In this case, using a volume rendering, the display control unit 9 forms a display image as it is for a region retrieved by the region retrieval unit 8 from the volume data stored in the data storage area 6. Furthermore, if the direction in which a volume rendering image is observed has been specified in advance, the display control unit 9 forms the volume rendering image as viewed from this direction. For instance, where an organ to be examined is a fetus and the fetus's face is an observation target region name, the region retrieval unit 8 acquires the location of the fetus's face from the volume data. Then, the display control unit 9 volume-renders only the region acquired by the region retrieval unit 8 and thereby outputs (i.e., displays) a display image onto the output unit 4.

Here, a method in which an operator specifies a region name, thereby acquiring a display image as described above, is called “a region name specifying mode.” Meanwhile, as described above, an operator may require a representative sectional image. For example, according to the technical document described above, a representative sectional image can automatically be captured from the volume data by specifying a representative section name. Here, such a method in which a display image is determined by the name of a representative section is called “a section name specifying mode.” Where the display control unit 9 is also able to form a display image by specifying the representative section name, the input unit 1 may include a switch for switching between the region name specifying mode and the section name specifying mode. This enables an operator to switch between both modes by selecting the mode required. Such a switch may be produced, for example, by disposing buttons on the apparatus, virtual buttons appearing on a touch panel, or by voice input.

As described above, in an ultrasound diagnostic apparatus according to the first embodiment, the optimal two-dimensional image used for observing a region of the organ to be examined can be formed from the volume data and displayed simply by specifying this region name, thus enabling the examination of the region without intuitive operation or complex procedures.

Next will be described a second embodiment. In the first embodiment, a region name specified by an operator is received and a display image corresponding to this region is formed. In the second embodiment, after receivable region names are retrieved from the volume data and they are presented, a region name specified by an operator is received and a display image corresponding to this region is formed. Only those features of the configuration of an ultrasound diagnostic apparatus according to the present embodiment that are different from those of the first embodiment will be described. The other features of the present embodiment are identical to those of the first embodiment; therefore, compositional elements or the like identical to those in the first embodiment are labeled with identical reference numerals, and the explanations described above are shared and omitted here.

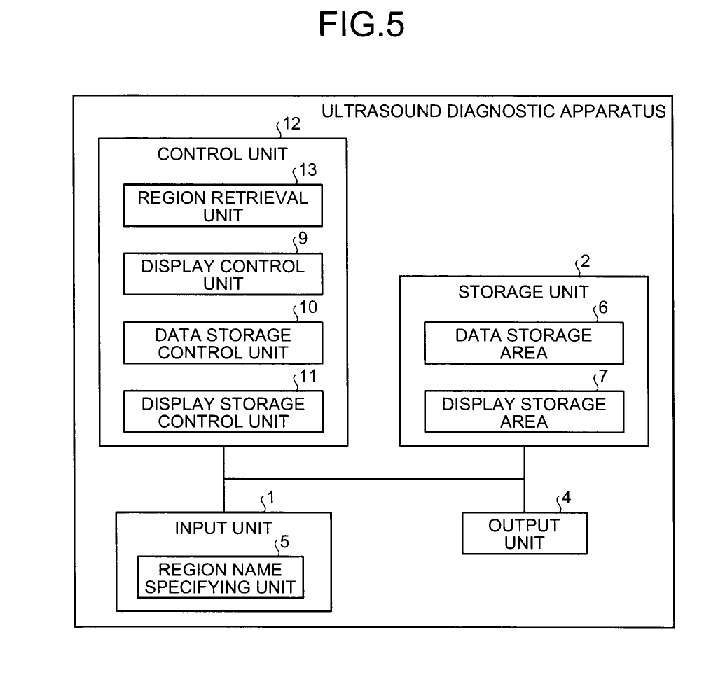

As shown in FIG. 5, the ultrasound diagnosis apparatus according to the second embodiment includes an input unit 1, a storage unit 2, a control unit 12, and an output unit 4. These units are electrically or electronically coupled together, and are connected by circuits that mutually exchange control signals, information, etc.

The control unit 12 controls the overall ultrasound diagnostic apparatus and performs various data processing, operation, or calculation. The control unit 3 includes a region retrieval unit 13, the display control unit 9, the data storage control unit 10, and the display storage control unit 11. The control unit 12 is, for example, a processor.

The region retrieval unit 13 retrieves region receivable by the region name specifying unit 5, from three-dimensional volume data for organs stored in the data storage area 6. The method for retrieving a region may adopt, for example, the technology described in “Fast Automatic Heart Chamber Segmentation from 3D CT Data Using Marginal Space Learning and Steerable Features,” by Yefeng Zheng, et. al, Eleventh IEEE International Conference on Computer Vision 2007.

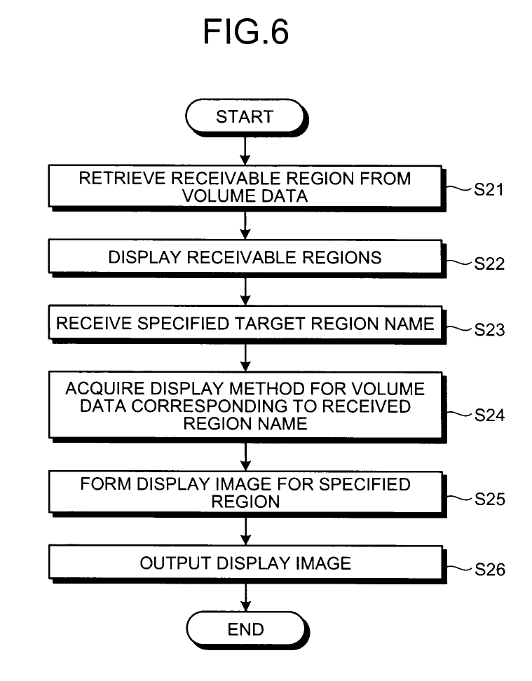

The operation of an ultrasound diagnostic apparatus according to the second embodiment will now be described with reference to FIG. 6. First, the region retrieval unit 13 retrieves region receivable by the region name specifying unit 5 from the three-dimensional volume data stored in the data storage area 6 (step S21). Where the organ corresponding to the volume data is the heart, the region retrieval unit 13 retrieves receivable regions from the volume data for the left ventricle, right ventricle, left atrium, right atrium, and aorta.

Subsequently, the region retrieval unit 13 outputs (i.e., displays) the receivable regions thus retrieved to the output unit 4 (step S22).

Next, the region name specifying unit 5 receives a target region name specified by an operator from among the group of receivable regions displayed on the output unit 4 (step S23). For example, where the operator needs to diagnose a disease of an aortic valve and the item “aortic valve” is displayed among the group of receivable regions, the aortic valve is specified.

Next, the display control unit 9 acquires from the display storage area 7 a display method for the volume data corresponding to the region name received by the region name specifying unit 5 (step S24).

Subsequently, the display control unit 9 captures volume data stored in the data storage area 6, a display method stored in the display storage area 7, and information about the region retrieved by the region retrieval unit 13. Then, according to this display method, the display control unit 9 forms a display image for the specified region (step S25).

In the second embodiment, based on the rules F6 and F7 assigned to the fist and second display methods respectively to be used where the item “aortic valve” is specified, the display control unit 9 forms the corresponding display images. In the rule F7, the sectional image most similar to dictionary B's is retrieved and displayed. However, it may easily be assumed that there may be some degree of variation in terms of the optimal sectional image for examination, depending on accuracy in the search for a sectional image, operator knowledge and experience, or simple preference. To overcome such drawbacks, the input unit 1 may incorporate the capacity to make fine adjustments to the sectional images acquired. Examples of methods for actualizing this fine adjustment function include, for example, a method in which the direction of a normal line defining a sectional image can be adjusted by a knob, and a method in which not only the sectional image most similar to dictionary B's but also a plurality of sectional images merely very similar to dictionary B's are prepared in advance and an operator is allowed to select the optimal from among them.

Lastly, the display control unit 9 outputs (i.e., displays) the images for display thus formed to the output unit 4 (step S26).

Thus, in response to an operator's specifying a required region name from among a group of regions observable by the ultrasound diagnostic apparatus, the optimal image including this region is displayed on the output unit 4.

As described above, the ultrasound diagnostic apparatus according to the second embodiment retrieves receivable regions from the volume data in advance, presents them, receives a region name from among them, and forms from the volume data the optimal two-dimensional image for the observation of this region. This allows examination of the region without intuitive operation or complex procedures.

In the first and second embodiments, descriptions have been given using as an example an ultrasound diagnostic apparatus that includes an input unit, a storage unit, a control unit, and an output unit. The ultrasound diagnostic apparatus may be further configured as an ultrasound diagnostic system in which the input unit, the storage unit, the control unit, and the output unit are connected together by a network such as the Internet.

For example, the ultrasound diagnostic apparatus may use as basic hardware a general-purpose computer that includes a control device, a storage device, an external a storage device, a display device, and an input device. In other words, the region retrieval unit, the display control unit, the data storage control unit, and the display storage control unit can be actualized by programs executed by the processor in the computer. In this case, the ultrasound diagnostic apparatus may be actualized by installing the aforementioned programs in the computer in advance or by storing the programs on a recording medium, such as a CD-ROM, and installing the program in the computer from this recording medium as required. In addition, the storage unit can be actualized by suitable use of a storage medium such as the memory incorporated in or externally attached to the computer, a hard disk, CD-R, CD-RW, DVD-RAM, or DVD-R.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound diagnostic apparatus comprising:
a first storage unit configured to store three-dimensional image data acquired by measuring the organ;
a second storage unit configured to store a region name in association with a display method, the region name being the name of a region of the organ and the display method being for displaying the region of the organ;
a region retrieval unit configured to retrieve, based on the region name, the region from the three-dimensional image data; and
a display unit configured to display a two-dimensional image data; and
a display control unit configured to transform, based on the region retrieved by the region unit, the three-dimensional image data of the region into the two-dimensional image for the region and configured to display the two-dimensional image on the display unit using the display method.

2. The apparatus according to claim 1, further comprising a region name specifying unit that receives the region name specified, wherein
the region retrieval unit retrieves the region from the three-dimensional image data based on the received region name.

3. The apparatus according to claim 2, wherein
the region retrieval unit further retrieves receivable regions from the three-dimensional image data, and
the region name specifying unit receives the region name specified from the retrieved regions.

4. The apparatus according to claim 2, wherein
the second storage unit stores a plurality of combinations of the region names in association with the display method, and
the region name specifying unit receives the combinations of the region names specified.

5. The apparatus according to claim 1, wherein
the two-dimensional image is a sectional image of the three-dimensional image data.

6. The apparatus according to claim 1, wherein
the two-dimensional image is a volume rendering image of the three-dimensional image data.

7. An ultrasound diagnostic method, comprising:
retrieving, based on a region name which is the name of a region of an organ, the region from three-dimensional image data about the organ, the three-dimensional image data being acquired by measuring the organ; and
transforming, based on the retrieved region, the three-dimensional image data of the region into a two-dimensional image for the region; and
displaying the two-dimensional image on a display unit using a display method corresponding to the region.

8. A computer readable product having a computer readable medium including programmed instructions that, when executed by a computer, cause the computer to perform:
retrieving, based on a region name which is the name of a region of an organ, the region from three-dimensional image data about the organ, the three-dimensional image data being acquired by measuring the organ; and
transforming, based on the retrieved region, the three-dimensional image data of the region into a two-dimensional image for the region; and
displaying the two-dimensional image on a display unit using a display method corresponding to the region.
